(19) United States
(12) Patent Application Publication
(10) Pub. No.: US 2022/0073966 A1
(43) Pub. Date: Mar. 10, 2022

(54) VIAL CAPS FOR BIOLOGICAL PROCESSING OR ANALYSIS

(71) Applicant: Biomeme, Inc., Philadelphia, PA (US)

(72) Inventors: Marc DeJohn, Philadelphia, PA (US); Jesse Wilson vanWestrienen, Philadelphia, PA (US); Christopher Cox, Elkins Park, PA (US); Paul Parkhurst, Philadelphia, PA (US)

(21) Appl. No.: 17/473,220

(22) Filed: Sep. 13, 2021

Related U.S. Application Data
(63) Continuation of application No. PCT/US2020/022368, filed on Mar. 12, 2020.
(60) Provisional application No. 62/942,320, filed on Dec. 2, 2019.
(60) Provisional application No. 62/818,297, filed on Mar. 14, 2019.

(51) Int. Cl. C12Q 1/686; B01L 3/00; B65D 39/04

(52) U.S. Cl. CPC C12Q 1/686; B01L 3/50825; B01L 3/50853; B01L 2300/0848; B01L 2300/123; B01L 2200/0689; B01L 2300/046; B65D 39/04

(57) ABSTRACT

Provided herein are vial caps for use with assay vials, tubes, or plates. The vial caps can be compatible with various analytic devices, for example, thermocycler. The vial caps can be used with polymerase chain reaction (PCR) assay vials, tubes, or plates in any thermocycling reactions. The vial caps described herein can prevent evaporation during thermocycling reactions.

### CROSS-REFERENCE

This application is a continuation of International Application No. PCT/US20/22368 filed Mar. 12, 2020 which claims priority to U.S. Provisional Patent Application No. 62/818,297, filed Mar. 14, 2019, and U.S. Provisional Patent Application No. 62/942,320, filed Dec. 2, 2019, each of which is entirely incorporated herein by reference.

### BACKGROUND

Caps can be used with sample vials during thermocycling reactions. Current solutions provide for plastic vials may include shallow caps which can provide containment of reagents but may not prevent condensation on cooler surfaces of the plastic vial which lie beyond the thermocycling heating elements of most thermocyclers. Loss of liquid volume in this way can cause concentration changes in the liquid analyte which can interfere with the progression of thermocycling reactions such as polymerase chain reaction (PCR) chemistry and fluorescence measurements.

### SUMMARY

A more advantageous apparatus and methods of use are described herein.

In an aspect, the present disclosure provides a vial cap for sealing a tube for processing a biological sample, comprising a top surface and a protrusion extending from the top surface, wherein the protrusion has a length of at least 5 millimeters, wherein the vial cap is configured such that when the vial cap seals the tube, (i) the protrusion extends into the tube along a length of the tube, and (ii) a ratio of the length of the protrusion to the length of the tube is less than 1:1.

In some embodiments, the vial cap comprises a polymeric material. In some embodiments, the polymeric material is an elastomeric material. In some embodiments, the elastomeric material is santoprene, resin, polypropylene or silicone. In some embodiments, the vial cap comprises an additive. In some embodiments, the additive is a color concentrate. In some embodiments, the top surface of the vial cap comprises a recessed region. In some embodiments, the protrusion comprises a bottom surface, wherein the bottom surface comprises a collapsing cavity extending into the vial cap from the bottom surface. In some embodiments, the ratio is at most about 0.9:1, or at most 0.7:1. In some embodiments, the ratio is at most about 0.5:1. In some embodiments, the vial cap is configured to seal the tube having a volume of at most about 300 microliters. In some embodiments, the protrusion comprises a bottom surface having a width, and wherein a ratio of the length of the protrusion to the width is at least 1.5:1. In some embodiments, the ratio is at least 2:1.

In another aspect, the present disclosure provides a vial cap for sealing a tube for processing a biological sample, comprising a top surface and a protrusion extending from the top surface, wherein the protrusion has a length, wherein the vial cap is configured such that when the vial cap seals the tube, (i) the protrusion extends into the tube along a length of the tube, and (ii) a geometric ratio of the length of the protrusion to the length of the tube is selected to operatively optimize a utility of the vial cap during a reaction. In some embodiments, the geometric ratio of the length of the protrusion to the length of the tube is less than 1:1.

In another aspect, the present disclosure provides a method for processing a biological sample, comprising: (a) providing a tube comprising the biological sample, wherein the tube is sealed by a cap comprising a top surface and a protrusion extending from the top surface into the tube, wherein the protrusion has a length of at least 5 millimeters, wherein the cap extends into the tube along a length of the tube, and wherein a ratio of the length of the protrusion to the length of the tube is less than 1:1; and (b) with the cap sealing the tube, subjecting the biological sample in the tube to processing.

In some embodiments, (b) comprises subjecting the biological sample to conditions sufficient for a polymerase chain reaction. In some embodiments, the tube further comprises a solution comprising the biological sample, and wherein in (b) a bottom surface of the protrusion is separated from a surface of the solution by a gap. In some embodiments, the gap has a length of at most about 5 millimeters. In some embodiments, a ratio of a length of the gap to the length of the tube is at most about 0.3:1.

In another aspect, the present disclosure provides a method to optimize an operation of a reaction, comprising: providing a vial cap for sealing a tube, comprising a top surface and a protrusion extending from the top surface, wherein the protrusion has a length, and wherein the vial cap is configured such that when the vial cap seals the tube, (i) the protrusion extends into the tube along a length of the tube and (ii) a geometric ratio of the length of the protrusion to the length of the tube is selected to operatively optimize a utility of the vial cap during the reaction.

In some embodiments, the geometric ratio of the length of the protrusion to the length of the tube is less than 1:1. In some embodiments, the method further comprises preparing the tube for the reaction by filling the tube with a sample that is subject to the reaction. In some embodiments, the method further comprises affixing the vial cap on the tube to create a seal between the vial cap and the tube.

In another aspect, the present disclosure provides a method for processing or analyzing a biological sample, comprising: (a) providing a tube comprising a solution comprising the biological sample; (b) sealing the tube with a vial cap comprising a top surface and a protrusion extending from the top surface into the tube; (c) subjecting the solution to conditions sufficient to perform a chemical or biological reaction on the biological sample, which chemical or biological reaction generates a signal in the solution; and (d) detecting at least about 80% of the signal from the solution.

In some embodiments, the chemical or biological reaction is a polymerase chain reaction. In some embodiments, the chemical or biological reaction is an isothermal reaction. In some embodiments, the protrusion has a length of at least 5 millimeters. In some embodiments, a ratio of the length of the protrusion to a length of the tube is less than 1:1. In some embodiments, a bottom surface of the protrusion is separated from a surface of the solution by a gap. In some embodiments, the gap has a length of at most about 5 millimeters. In some embodiments, a ratio of the length of the gap to the length of the tube is at most about 0.3:1.

In another aspect, the present disclosure provides a method for processing or analyzing a biological sample, comprising: (a) providing a tube comprising a solution comprising the biological sample; (b) sealing the tube with a vial cap comprising a top surface and a protrusion extending from the top surface into the tube; wherein in (b) a bottom surface of the protrusion is separated from a surface of the solution by a gap comprising a vapor phase, and wherein a ratio of a length of the protrusion to a length of the tube is such that a partial pressure of a species from the solution in the vapor phase is less than 1 atm at a temperature of 25° C.

In an illustrative use of the herein described vial cap, the vial cap is placed snug onto the PCR tube upon the preparation of the PCR tube for use in a selected PCR analysis protocol. Illustratively, the preparation of the PCR tube can comprise removing a plastic film or foil present on the PCR tube operative to preserve the sterility of volume of the PCR tube or to retain the sample inside the PCR tube and filling the PCR tube with a selected sample subject of the PCR analysis.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a strip of sample vials 101 sealed with a strip of void filling caps 102 that can be used to hold samples for reactions such as thermocycling reactions. In FIG. 1, three connected sample vials and three connected void filling caps are shown. Each individual sample vial 103 is sealed with a void filling cap 104. The void filling cap 104 comprises a top surface 105 having a recessed region 106. The void filling caps are connected by a surface 107.

FIG. 2 shows a perspective view of a strip of sample vials 201 sealed with a strip of void filling caps 202. Three sample vials and three void filling caps are shown. The sample vials can be plastic vials. The void filling caps can comprise elastomers. Each void filling cap has a top surface 203 and a protrusion 204 extending from the top surface 203. The protrusion is inserted into the sample vial to seal the sample vial. A seal region 205 is generated when the protrusion 204 is inserted into the sample vial and the bottom portion 206 of the protrusion is in contact with the inner wall of the sample vial.

FIG. 3 shows a section review of a strip of sample vials 301 sealed with a strip of void filling caps 302. A sample vial 303 contains a liquid sample 304. A void filling cap comprises a top surface 305 and a protrusion 306 extending from the top surface 305. The bottom surface of the protrusion comprises a collapsing hole (or collapsing cavity) 307. The collapsing hole 307 of the protrusion 306 can allow for compression inwards of the material of the protrusion 306 as it makes contact and is forced downward into the sample vial to form a tight seal. The collapsing hole 307 can prevent the void filling cap from being pushed outward from the sample vial.

FIG. 4 shows a vertical cross section view of a strip of sample vials 401 sealed with a strip of void filling caps 402. Each sample vial contains a liquid sample 403. When sealing the sample vial with the void filling cap, an air space (or gap region) 404 is formed in between the bottom of the protrusion 405 of the void filling cap and the liquid sample. The protrusion 405 comprises a taper transition region 406 immediately adjacent to the top surface. The protrusion 405 further comprises a tapered region (e.g., the cylinder portion) 407 immediately following the taper transition region 406.

FIG. 5 shows example PCR data comparing reactions performed with void filling cap and reactions performed with mineral oil. The fluorescent dye used in the reactions was FAM.

FIG. 6 shows example PCR data comparing reactions performed with void filling cap and reactions performed with mineral oil. The fluorescent dye used in the reactions was Texas Red X.

FIG. 7 shows example PCR data comparing reactions performed with void filling cap and reactions performed with mineral oil. The fluorescent dye used in the reactions was ATTO647N.

FIG. 8 shows example PCR data comparing reactions performed with white void filling cap and reactions performed with mineral oil. The fluorescent dye used in the reactions was FAM.

FIG. 9 shows example PCR data comparing reactions performed with white void filling cap and reactions performed with mineral oil. The fluorescent dye used in the reactions was Texas Red X.

FIG. 10 shows example PCR data comparing reactions performed with white void filling cap and reactions performed with mineral oil. The fluorescent dye used in the reactions was ATTO647N.

FIGS. 11A-11D show dimensions of example vial caps and parts thereof. The length of each part of the vial caps and the surface connecting the vial caps are shown in millimeters. FIG. 11A shows an example view from the top of a strip of three void filling caps with measurements showing the length and width of the strip and parts thereof. FIG. 11B shows a vertical cross section view (e.g., section A-A as indicated in FIG. 11A) of a void filling cap. FIG. 11C shows a side view of a strip of three void filling caps with measurements showing the length of each vial cap and parts thereof. FIG. 11D shows an example view from the bottom of the void filling cap.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed. It is appreciated that although the vial caps are described in the Figures as having a configuration comprising three void filling caps filling three vials in linear arrangement, that such description is merely illustrative as the inventive concepts described herein contemplate various configurations and numbers of void filling caps.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, or within 2-fold, of a value. Where particular values are described in the application and claims, unless otherwise stated the term “about” meaning within an acceptable error range for the particular value may be assumed.

### Overview

Thermocycling small volumes of liquid samples may lead to sample loss due to evaporation. Caps can be used with sample tubes or vials to allow continued use of thermocyclers while avoiding problems associated with thermocycling small volumes of liquid samples. The caps can be used with a tube. The caps can be used with a tube for sample processing. The caps can be used with polymerase chain reaction (PCR) sample tubes, vials or plates, for example, standard sized conical PCR vials. However, using standard caps may not prevent condensation on cooler surfaces of the plastic vial, which can still lead to concentration changes in the liquid samples.

To address this problem, some instrument designs can employ a lid which applies heat to the upper portions of the plastics. While these heated lids may not thermocycle, they may prevent dew formation in standard plastic vials and caps.

In some cases, an instrument with heated lid is unavailable. For example, some instruments can be battery powered devices and for reasons of energy efficiency, among others, may have eschewed a heated-lid design. With no heated lid, a barrier may be used to prevent water vapor from escaping into upper portions of a plastic vial where condensation can accumulate and remain throughout the duration of an experiment. In some cases, oils and waxes can be used to form a vapor barrier over the liquid sample being heated. However, the use of oils and waxes may pose challenges to manufacturing, shipping and handling. Oils tend to migrate, escape packaging, and can interfere with other reagents stored in common volumes. Wax can be difficult to deliver and can cause problems during melt under high-heat storage conditions.

To improve the design, the present disclosure provides caps to be used with sample containers (e.g., tubes, vials and plates) which can be more reliable and convenient for large scale manufacturing.

Caps described herein, termed “void filling caps,” can be elastomeric caps. The void filling caps can be used with tubes. The void filling caps can be used with the standard PCR vials. The caps can create a seal near the surface of a predetermined fluid volume while filling the void in the standard vial above the heated fluid and preventing vapor from escaping into cooler portions of the vial where condensation can occur.

Example Tests show that a seal can be maintained with a small vapor volume between the end of the cap and liquid surface without condensation losses. Using the caps described herein during PCR reactions, PCR data can be equal to or higher quality compared with other vapor barriers (e.g., oil or wax).

### Void Filling Caps

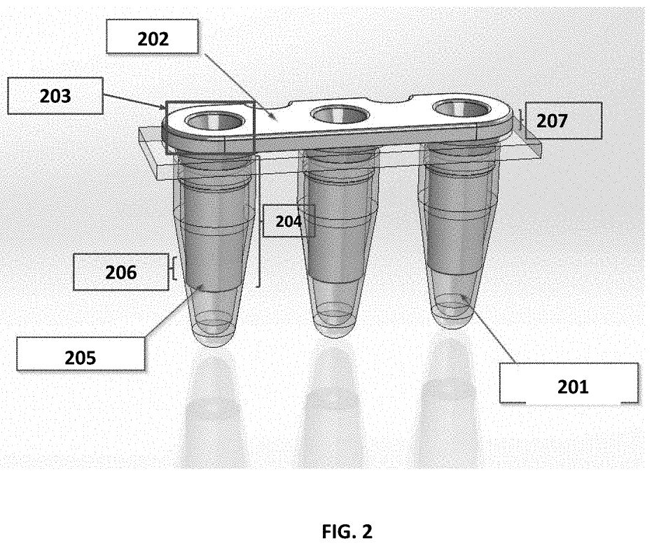

The void filling cap described herein can comprise a top surface and a protrusion extending from the top surface. The protrusion can be inserted into a sample vial to seal the sample vial. For example, as shown in FIG. 2, each void filling cap has a top surface 203 and a protrusion 204 extending from the top surface 203. In this example, the protrusion is inserted into the sample vial to seal the sample vial. A seal region 205 can be generated when the protrusion 204 is inserted into the sample vial and the bottom portion 206 of the protrusion is in contact with the inner wall of the sample vial.

The top surface of the void filling cap can be in various shapes or configurations. The void filling cap can be an individual cap, which can be used to seal an individual vial or tube. In some cases, multiple void filling caps can be connected to form a strip of void filling caps. The strip of void filling caps may be used to seal a strip of sample vials or tubes. The strip of void filling caps may comprise at least 2, 3, 4, 5, 6, 7, 8, 9, 10, 11, 12, 13, 14, 15, or more void filling caps. In some cases, multiple void filling caps can be connected to form an array of void filling caps. The array of void filling caps may be used with an array of sample vials or tubes. The assay of void filling caps may comprise at least 3, 4, 5, 6, 7, 8, 9, 10, 20, 40, 60, 70, 80, 100, or more void filling caps. For example, the void filling caps can be used to seal a 8-tube strip, 12-tube strip, 24-well plate, 32-well pate, 48-well plate, 56-well plate, 64-well plate, 72-well plate, 80-well plate, or 96-well plate. In some cases, the void filling cap can be connected to form a mat, which can be used to seal multi-well plates. The void filling caps can be compatible with an analytic device, for example, a thermocycler.

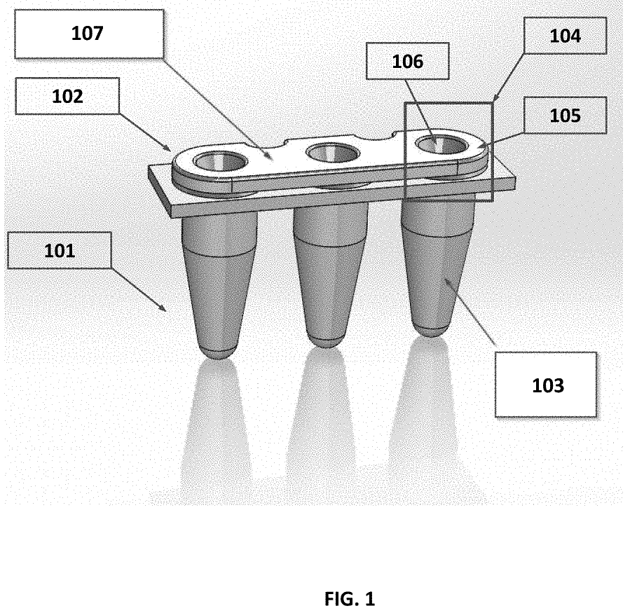

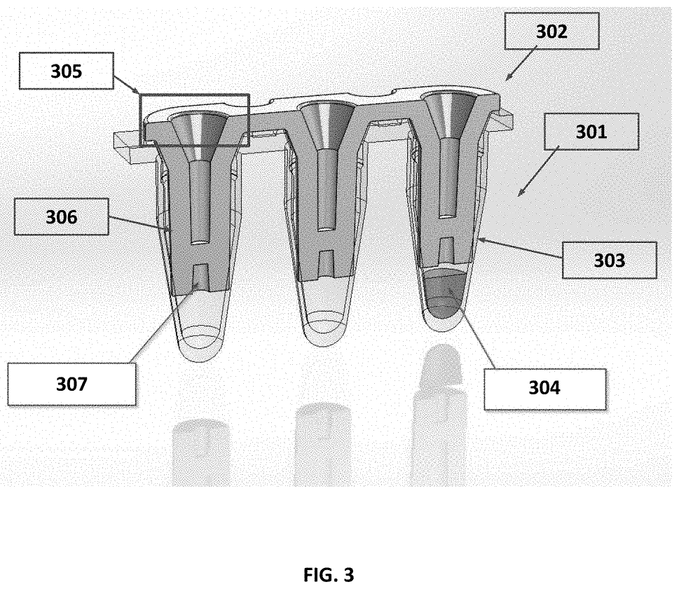

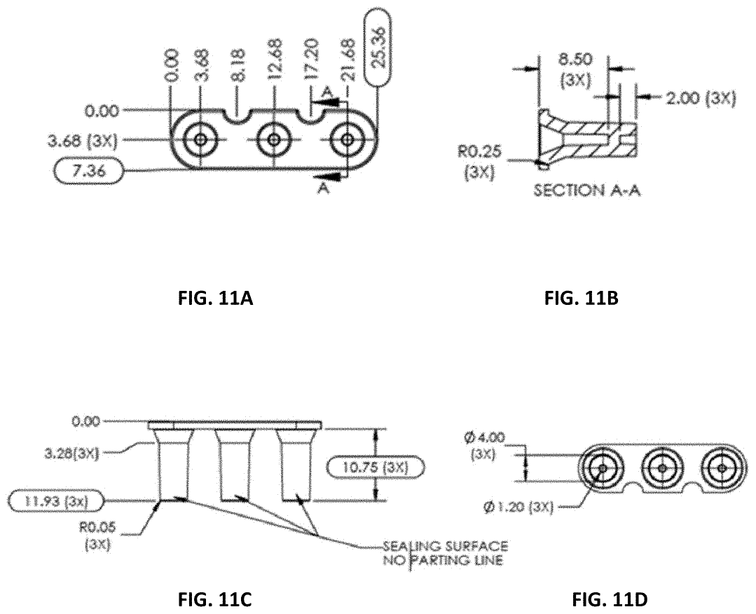

The void filling cap provided herein can be used for sealing a tube for sample processing such as a polymerase chain reaction (PCR) tube. The tube may be used to perform a chemical or biological reaction, such as, nucleic acid extension or amplification (e.g., polymerase chain reaction or isothermal amplification). The void filling cap can comprise a top surface and a protrusion extending from the top surface. The protrusion can be at least about 5 millimeters (mm) in length. FIG. 11A shows an example view from the top of a strip of three void filling caps with measurements showing the length and width of the strip and parts thereof. The strip of three void filling caps are measured about 25.36 mm long and 7.36 mm wide. FIG. 11B shows a vertical cross section view (e.g., section A-A as indicated in FIG. 11A) of a void filling cap. The void filling cap can comprise a recessed region (e.g., 106 of FIG. 1) extending to the protrusion such that a part of the protrusion has a hollow center. FIG. 11B shows that an example length of the recessed region is about 8.5 mm. In some cases, the recessed region may be at least about 1.0 mm, 1.5 mm, 2.0 mm, 2.5 mm, 3.0 mm, 3.5 mm, 4.0 mm, 4.5 mm, 5.0 mm, 5.5 mm, 6.0 mm, 6.5 mm, 7.0 mm, 7.5 mm, 8.0 mm, 8.5 mm, 9.0 mm, 9.5 mm, 10.0 mm, 10.5 mm, 11.0 mm, 11.5 mm, 12.0 mm, 12.5 mm, 13.0 mm, 13.5 mm, 14.0 mm, 14.5 mm or more. The recessed region may be extended to the bottom of the protrusion such that the void filling cap has a hollow center. The bottom surface of the protrusion may comprise a collapsing hole or collapsing cavity (e.g., 307 of FIG. 3). FIG. 11B shows an example length of the collapsing hole or collapsing cavity of about 2 mm. In some cases, the length of the collapsing hole or collapsing cavity may be at least about 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm or more. The top surface of the void filling cap can comprise a broken edge (e.g., a breaking edge). The radius of the broken edge shown in FIG. 11B is 0.25 mm.

The PCR tube can be a PCR microtube. For example, the PCR tube can have a volume of at most about 300 microliter (μL). The PCR tube can have a capacity to hold a liquid of equal to or at most about 300 μL, 250 μL, 200 μL, 180 μL, 150 μL, 100 μL, 90 μL, 80 μL, 50 μL or less. In some cases, the PCR tube may have a volume of at least about 300 μL. The PCR tube may have a capacity to hold a liquid of equal to or at least about 300 μL, 350 μL, 400 μL, 450 μL, 500 μL, 550 μL, 600 μL, 650 μL, 700 μL, 750 μL, 800 μL, 850 μL, 900 μL, 950 μL, 1,000 μL, 1,200 μL, 1,500 μL, 1,800 μL, 2,000 μL or more.

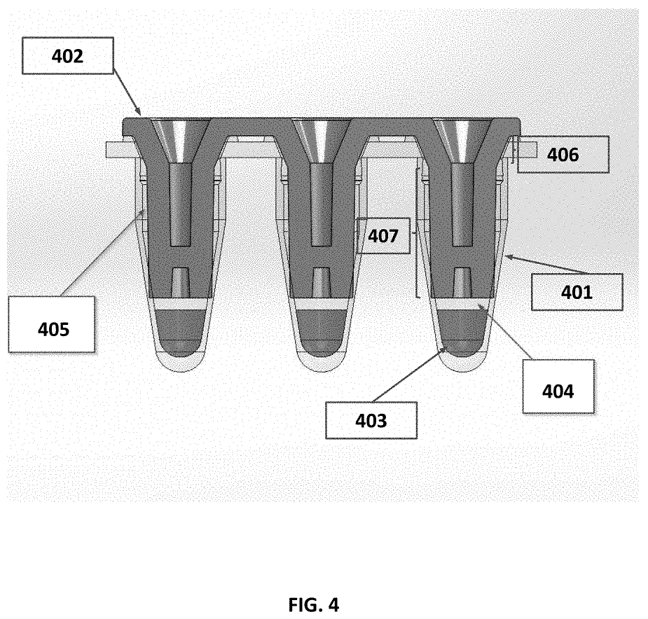

The protrusion can be at least about 5 mm in length, extending from the top surface. The top surface may have a thickness, and, in this case, the protrusion can be measured from the bottom of the top surface of the void filling cap to the bottom of the protrusion. For example, FIG. 2 shows the thickness 207 of the top surface 203. The top surface may have a thickness of at least about 0.1 mm, 0.2 mm, 0.3 mm, 0.4 mm, 0.5 mm, 0.6 mm, 0.7 mm, 0.8 mm, 0.9 mm, 1.0 mm, 1.2 mm or more. In some cases, the protrusion is at least about 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or more in length. FIG. 11C shows a side view of a strip of three void filling caps with measurements showing the length of each vial cap and parts thereof. The total length of each void filling cap measured from the top of the top surface to the bottom of the protrusion is about 11.93 mm. The protrusion has a length of about 10.75 mm measured from the bottom of the top surface to the bottom of the protrusion. The thickness of the top surface is calculated to be about 1.18 mm. The protrusion can comprise a taper transition region (e.g., 406 of FIG. 4). The length of the taper transition region measured from the top of the top surface to the bottom of the taper transition region can be at least about 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm or more. For example, FIG. 11C shows the length of the taper transition region is about 3.28 mm. The bottom surface of the protrusion may comprise a broken edge (e.g., a breaking edge). The radius of the broken edge shown in FIG. 11C is 0.05 mm.

The protrusion can comprise a tapered region (e.g., 407 of FIG. 4). The horizontal cross section of the tapered region of the protrusion may be in a circular configuration and may have a diameter of at least about 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.5 mm, 4.6 mm, 4.7 mm, 4.8 mm, 4.9 mm, 5.0 mm or more. FIG. 11D shows an example view from the bottom of the void filling cap. The diameter of the tapered region of the protrusion in this example is about 4 mm. The diameter of the collapsing hole or collapsing cavity of the protrusion in this example is about 1.2 mm.

The void filling cap can be configured such that when the void filling cap seals the PCR tube, a ratio of the length of the protrusion to a length of the PCR tube may be less than 1:1. The ratio of the length of the protrusion to the length of the PCR tube may be at most about 0.9:1, 0.8:1, 0.7:1, 0.6:1, 0.5:1 or less. It is to be understood that when the void filling cap seals the PCR tube, the bottom of the top surface of the void filling cap and the top of the PCR tube can be immediately adjacent to each other. In such case, the length of the protrusion measured from the bottom of the top surface of the void filling cap may be approximately equal to the length measured from the top of the PCR tube. For the purpose of determining the ratios described herein, the length of the protrusion or the length of the PCT tube are measured from the bottom of the top surface of the top surface of the void filling cap. In some cases, the ratio of the length of the protrusion to a length of the PCR tube may be at most about 0.9:1, 0.8:1, 0.7:1, 0.6:1, 0.5:1, 0.4:1, or less. In some cases, the ratio of the length of the protrusion to a length of the PCR tube may be at least about 0.4:1, 0.5:1, 0.6:1, 0.7:1, 0.8:1 or more. The PCR tube described herein can be the standard PCR microtube with a length of about 15 to 21 mm and a volume capacity of about 150-300 μL.

An air space or gap region can be generated between the bottom of the protrusion and the surface of a liquid sample (e.g., 404 of FIG. 4) when the void filling cap seals the PCR tube. The length of the gap region (measured from the bottom of the protrusion and the surface of the liquid sample) can be at most about 5.0 mm, 4.5 mm, 4.0 mm, 3.5 mm, 3.0 mm, 2.5 mm, 2.0 mm, 1.5 mm, 1.0 mm, 0.5 mm or less. A ratio of the length of the gap region to a length of the PCR tube can be at most about 0.3:1, 0.2:1, 0.1:1 or less.

### Materials

The void filling cap described herein can comprise a base material. The base material can be of various materials. In some cases, the base material of the void filling cap is a plastic material. In some cases, the base material of the void filling cap is an elastomeric material. The elastomeric material can be thermoplastic elastomers. The elastomeric material can be rubbery copolymer elastomers. Examples of rubbery copolymer elastomers include, but are not limited to, anionic polymerized olefinic elastomers. Examples of anionic polymerized olefinic rubbers include ethylene-propylene rubber, ethylene-propylene-diene monomer rubber, polyisobutylene, or “butyl rubber”, or any other polymer of isoolefin optionally copolymerized with conjugated diene (such as isoprene), optionally containing up to 30 wt. % or an α,β-ethylenic unsaturated nitrile and/or styrenic comonomer (such as styrene and/or alkyl substituted styrene), and the like. In some cases, the base material of the void filling cap is isobutylene-isoprene copolymer or isobutylene-para methylstyrene copolymer. In some cases, the base material of the void filling cap is santoprene (e.g., SANTOPRENE 8211-45 and SANTOPRENE 8211-65). In some cases, the base material of the void filling cap is resin, for example, FLFLGR02. In some cases, the base material of the void filling cap is silicon.

A wide variety of polymers and resins may be utilized to make the void filling cap. These include thermoplastic, thermosetting polymers and resins. Example polymers include polyolefins and olefin copolymers, polyesters, polyphenylene ether resins (PPO), polystyrene and styrene copolymers, polyamides, polyimides, polyurethanes, polyvinylchloride (PVC), acrylic resins, polycarbonates, ABS resins, polyvinylchloride, allyl polymers, epoxy resins, phenolic resins, thermosetting polyesters, urea and melamine formaldehyde resins. Examples of polyolefins and olefin copolymers include, for example, polyethylene, polypropylene, ethylene propylene copolymers, polybutylene, and EVA. Various forms of polyethylene can be utilized including low-density polyethylene, and high-density polyethylene. Examples of styrene copolymers include high impact polystyrene (HIPS), styrene-maleic anhydride copolymer (SMA), styrene-acrylonitrile copolymer (SAN), styrene-methylacrylate copolymers, styrene-butadiene or styrene-isoprene block copolymers or their hydrogenated versions. An example thermoplastic polyamide is nylon. Examples of polyesters are PET and PBT. Examples of PVC polymers include rigid PVC (Premium 1401-11N) and a rigid PVC blend available from Alcan containing 10% TiO₂.

The void filling cap can be flexible. The void filling cap can bend and compress. The elastomeric material can be soft or hard and can be of various durometer scales. For example, the ASTM D2240 standard recognizes twelve different shore durometer scales using combinations of specific spring forces and indentor configurations. These scales are referred to as durometer types, including durometer type A, C, D, B, M, E, R, O, OO, DO, OOO, and OOO-S. Each scale results in a value between 0 and 100, with higher values indicating a harder material. In some cases, the void filling cap comprises a medium durometer santoprene (e.g., 65 shore A).

### Additives

The void filling cap may further comprise an additive, for example, color concentrate. The color concentrate can be made by mixing a colorant with a carrier. In some cases, the carrier is a resin, e.g., ethylene-methyl acrylate (EMA). The colorant can be any color, e.g., white, red, orange, yellow, green, cyan, blue, purple, and black. An example of color concentrate is Linli color, LC2002 white universal 50/1 color concentrate.

When preparing a polymer composition to be used to make the cap, at least one polymer may be blended with the color concentrate. For example, the color concentrate may be blended into the polymer by mixing in a ribbon blender or tumble blender.

### Methods of Use

The caps described herein (e.g., void filling caps) can be compatible with various assays, for example, biological assays. The biological assays can include thermocycling assays, for example, polymerase chain reaction (PCR) assays, melting curve assays, isothermal assays or other assays that may comprise heating the assay tubes to certain temperatures. The void filling caps can be used with the standard PCR vials (e.g., PCR vial with capacity of 200 μL, 300 μL, 500 μL, 1.5 mL, or 2 mL). The caps can create a seal near the surface of a predetermined fluid volume while filling the void in the standard vial above the heated fluid and preventing vapor from escaping into cooler portions of the vial where condensation can occur. The cooler portion may have a temperature that is lower than the heated fluid or the air immediately adjacent to the heated fluid (e.g., the gap region). For example, the cooler portion may have a temperature that is at least about 5° C., 10° C., 15° C., 20° C., 25° C., 30° C., 35° C., 40° C., 45° C., 50° C., or more lower than the heated fluid.

The cap may reduce an amount of a solution that may evaporate and condense on a surface within the tube. The cap may reduce the condensation generated from a solution by at least 50%, 60%, 70%, 80%, 90%, 95% or more in comparison with the condensation generated from the solution using a cap without the protrusion.

The void filling cap can be equally or more effective in preventing or reducing evaporation in comparison with oil or wax.

The methods described herein can be used for processing a biological sample. For example, the method can comprise providing a tube comprising the biological sample. The tube can be sealed by a cap comprising a top surface and a protrusion extending from the top surface into the tube. The protrusion can have a length of at least 5 millimeters. The cap can extend into the tube along a length of the tube. A ratio of the length of the protrusion to the length of the tube may be less than 1:1. Next, with the cap sealing the tube, the biological sample in the tube can be subjected to processing.

For another example, the method described herein can comprise providing a tube comprising a solution comprising the biological sample. Next, the tube can be sealed with a vial cap comprising a top surface and a protrusion extending from the top surface into the tube. A bottom surface of the protrusion may be separated from a surface of the solution by a gap comprising a vapor phase. A ratio of a length of the protrusion to a length of the tube may be such that a partial pressure of a species from the solution in the vapor phase is less than 1 atm (i.e., 101.325 Kilopascal) at a temperature of 25° C.

The void filling cap may also provide potential optical benefits when used in concert with an assay device, for example, a thermocycler device. In some cases, the thermocycler device has a sensor and light path that is perpendicular to the PCR tube. And in such cases, when using the void filling cap disclosed herein, the light emitted in the PCR reaction can reflect off the void filling cap and make its way back down and through the PCR tube to the sensor configured perpendicular to the PCR tube. With clear vapor barriers such as mineral oil or wax, the light can escape through the barrier such that it can be out of the light path to the sensor. The void filling cap provided herein can minimize signal loss of a signal generated from a liquid sample. The signal loss may be minimized to at most about 40%, 35%, 30%, 25%, 20%, 15%, 10%, 5%, or less of said signal. The signal from the sample in the PCR tube can be detected by a detector during the PCR cycles. The detected signal can be at least about 60%, 65%, 70%, 75%, 80%, 85%, 90%, 95% or more of the signal originally generated from the sample in the PCR tube. The detected signal may be 100% of the signal originally generated from the sample in the PCR tube.

The methods can be used for processing or analyzing a biological sample. For example, the method can comprise providing a tube comprising a solution comprising the biological sample. Next, the tube can be sealed with a vial cap comprising a top surface and a protrusion extending from the top surface into the tube. Next, the solution can be subjected to conditions sufficient to perform a chemical or biological reaction on the biological sample. The chemical or biological reaction can generate a signal in the solution. Next, at least about 80% of the signal from the solution can be detected.

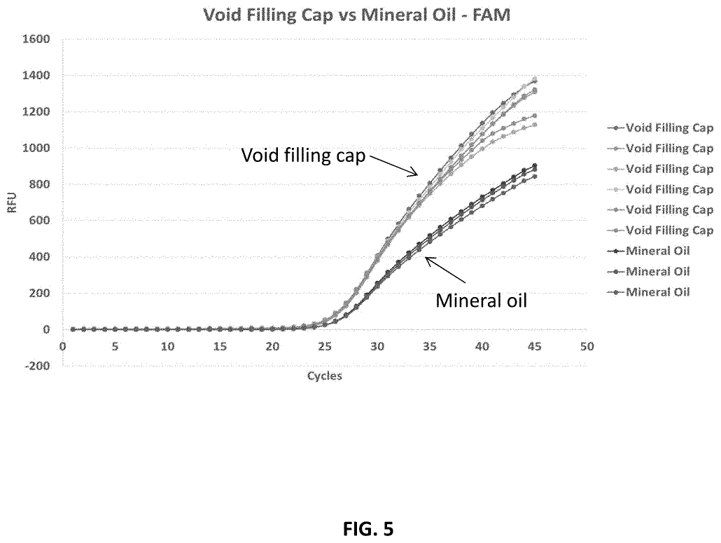

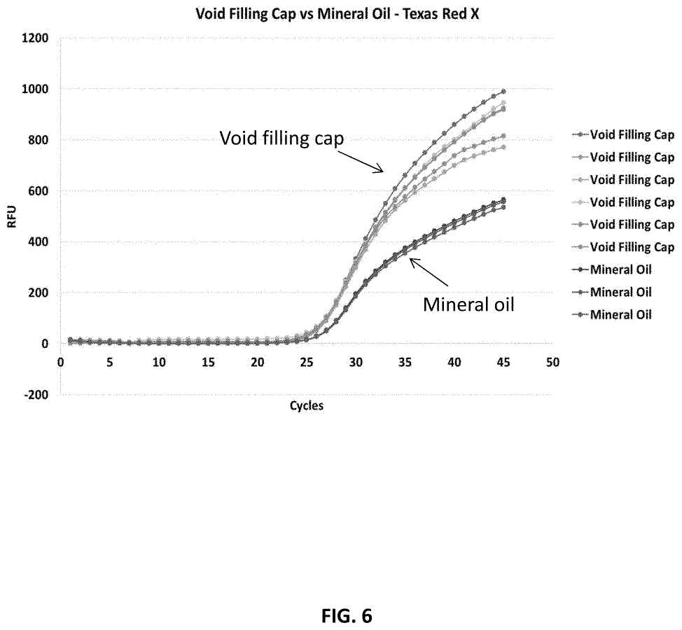

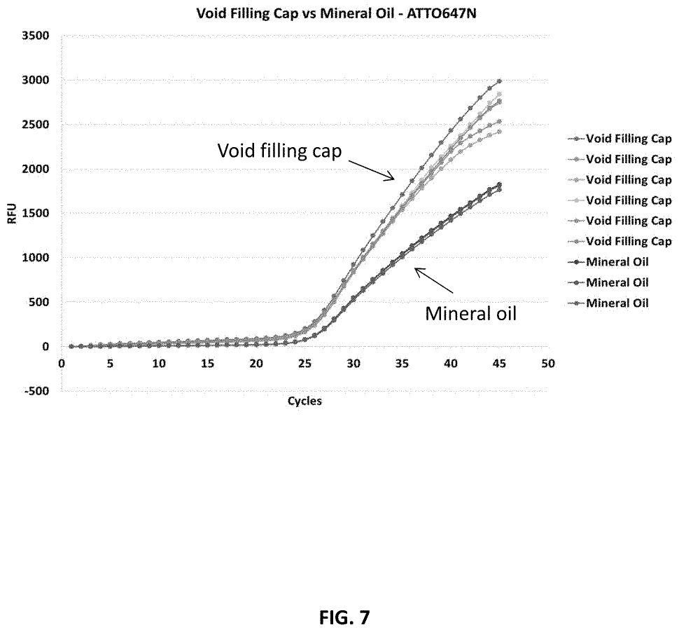

For example, FIG. 5 shows an example test comparing the qPCR results of assays performed with a medium durometer santoprene (e.g., 65 shore A) and mineral oil. The standard vial (BioPlastics 96 well format plate) and fluorescent dye FAM were used in the example test. The results showed that the assay group using void filling caps had lower Cycle quantification (Cq) values. The lower Cq values may be attributed to increased light levels, thus allowing the signal to emerge above background sooner. Similarly, FIG. 6 and FIG. 7 show example tests comparing void filling caps and mineral oil with different fluorescent dyes, Texas Red X and ATTO647N, respectively.

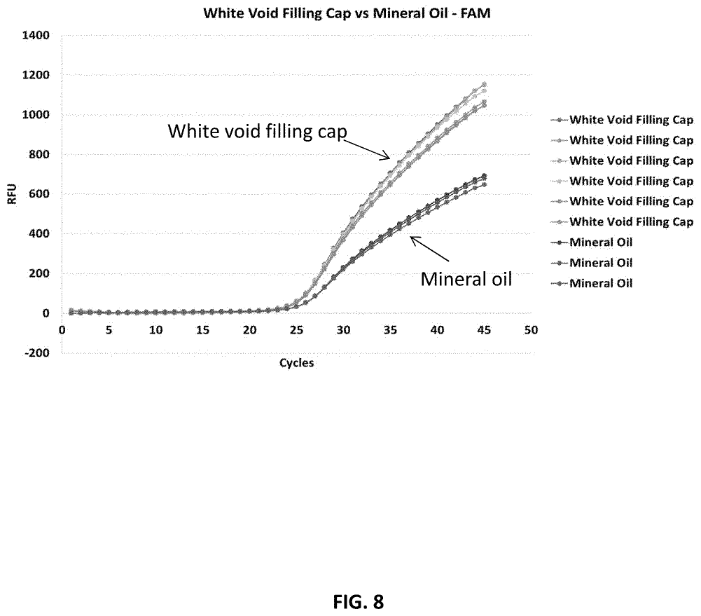

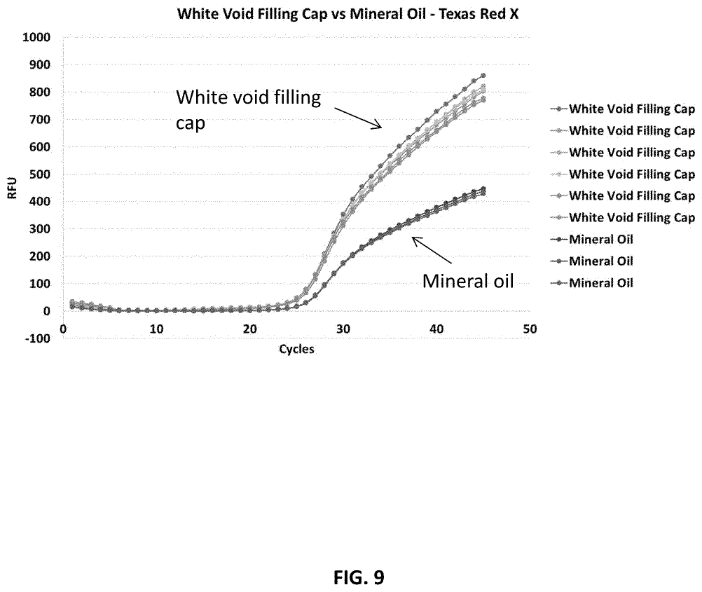

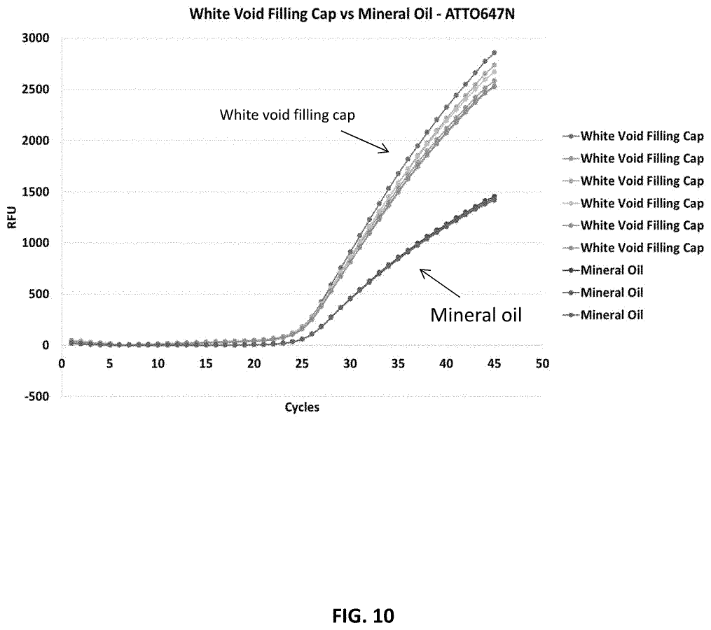

For another example, FIG. 8 shows an example test comparing the qPCR results of assays performed with a medium durometer santoprene (e.g., 65 shore A) with a white additive (e.g., Linli color, LC2002 white universal 50/1 color concentrate) and mineral oil. Similarly, FIG. 9 and FIG. 10 show example tests comparing white void filling caps and mineral oil with different fluorescent dyes, Texas Red X and ATTO647N, respectively.

Testing using void filling caps made of TPE SANTOPRENE 8211-45, FLFLGR02, polypropylene or silicone showed similar results, indicating equal or improved effect in providing optical benefits during thermocycling.

### Samples

A variety of samples (e.g., biological samples) may be analyzed in a PCR tube. A sample may be obtained invasively (e.g., tissue biopsy) or non-invasively (e.g., venipuncture). The sample may be an environmental sample. The sample may be a water sample (e.g., a water sample obtained from a lake, stream, river, estuary, bay, or ocean). The sample may be a soil sample. The sample may be a tissue or fluid sample from a subject, such as saliva, semen, blood (e.g., whole blood), serum, synovial fluid, tear, urine, or plasma. The sample may be a tissue sample, such as a skin sample or tumor sample. The sample may be obtained from a portion of an organ of a subject. The sample may be a cellular sample. The sample may be a cell-free sample (e.g., a plasma sample comprising cell-free analytes or nucleic acids). A sample may be a solid sample or a liquid sample. A sample may be a biological sample or a non-biological sample. A sample may comprise an in-vitro sample or an ex-vivo sample. Non-limiting examples of a sample include an amniotic fluid, bile, bacterial sample, breast milk, buffy coat, cells, cerebrospinal fluid, chromatin DNA, ejaculate, nucleic acids, plant-derived materials, RNA, saliva, semen, blood, serum, soil, synovial fluid, tears, tissue, urine, water, whole blood or plasma, and/or any combination and/or any fraction thereof. In one example, the sample may be a plasma sample that may comprise DNA. In another example, the sample may comprise a cell sample that may comprise cell-free DNA.

A sample may be a mammalian sample. For example, a sample may be a human sample. Alternatively, a sample may be a non-human animal sample. Non-limiting examples of a non-human sample include a cat sample, a dog sample, a goat sample, a guinea pig sample, a hamster sample, a mouse sample, a pig sample, a non-human primate sample (e.g., a gorilla sample, an ape sample, an orangutan sample, a lemur sample, or a baboon sample), a rat sample, a sheep sample, a cow sample, and a zebrafish sample.

The sample may comprise nucleic acids (e.g., circulating and/or cell-free DNA fragments). Nucleic acids may be derived from eukaryotic cells, prokaryotic cells, or non-cellular sources (e.g., viral particles). A nucleic acid may refer to a substance whose molecules consist of many nucleotides linked in a long chain. Non-limiting examples of the nucleic acid include an artificial nucleic acid analog (e.g., a peptide nucleic acid, a morpholino oligomer, a locked nucleic acid, a glycol nucleic acid, or a threose nucleic acid), chromatin, niRNA, cDNA, DNA, single stranded DNA, double stranded DNA, genomic DNA, plasmid DNA, or RNA. A nucleic acid may be double stranded or single stranded. A sample may comprise a nucleic acid that may be intracellular. Alternatively, a sample may comprise a nucleic acid that may be extracellular (e.g., cell-free). A sample may comprise a nucleic acid (e.g., chromatin) that may be fragmented.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1.-33. (canceled)

34. A method for processing a biological sample, comprising:
(a) providing a tube comprising a solution comprising said biological sample, wherein said tube is sealed by a cap comprising a top surface and a protrusion extending from said top surface into said tube, wherein said protrusion has a length of at least 5 millimeters, wherein said cap extends into said tube along a length of said tube, and wherein a ratio of said length of said protrusion to said length of said tube is less than 1:1; and
(b) with said cap sealing said tube, subjecting said biological sample in said tube to processing, wherein a bottom surface of said protrusion is separated from a surface of said solution by a gap.

35. The method of claim 34, wherein (b) comprises subjecting said biological sample to conditions sufficient for a polymerase chain reaction.

36. The method of claim 34, wherein said gap comprises a vapor phase.

37. The method of claim 36, wherein said gap has a length of at most about 5 millimeters.

38. The method of claim 36, wherein a ratio of a length of said gap to said length of said tube is at most about 0.3:1.

39. The method of claim 34, wherein said cap comprises a polymeric material.

40. The method of claim 39, where said polymeric material is an elastomeric material.

41. The method of claim 40, wherein said elastomeric material is santoprene, resin, polypropylene or silicone.

42. The method of claim 34, wherein said cap comprises an additive.

43. The method of claim 42, wherein said additive is a color concentrate.

44. The method of claim 34, wherein said top surface of said cap comprises a recessed region.

45. The method of claim 34, wherein said bottom surface comprises a collapsing cavity extending into said cap from said bottom surface.

46. The method of claim 34, wherein said ratio is at most about 0.9:1.

47. The method of claim 34, wherein said ratio is at most about 0.5:1.

48. The method of claim 34, wherein said cap is configured to seal said tube having a volume of at most about 300 microliters.

49. The method of claim 34, wherein said bottom surface has a width, and wherein a ratio of said length of said protrusion to said width is at least 1.5:1.

50. The method of claim 49, wherein said ratio is at least 2:1.

51. The method of claim 34, wherein said cap reduces condensation from said solution by at least about 50% compared with condensation from a solution generated in said tube with a cap without said protrusion.

52. The method of claim 51, wherein said biological sample generates a signal during said PCR.

53. The method of claim 52, wherein said cap reduces signal loss from said biological sample, and wherein a detected signal is at least about 80% of said signal generated from said biological sample.
